(19) United States
(12) Patent Application Publication
(10) Pub. No.: US 2025/0375325 A1
(43) Pub. Date: Dec. 11, 2025

(54) NONWOVEN MATERIALS WITH VARIABLE FIBER TO FIBER BOND DENSITY

(71) Applicant: The Procter & Gamble Company, Cincinnati, OH (US)

(72) Inventors: Gueltekin ERDEM, Cincinnati, OH (US); Joseph Henry NURRE, West Chester, OH (US); Zhiwei SHI, Cincinnati, OH (US)

(21) Appl. No.: 19/221,962

(22) Filed: May 29, 2025

Related U.S. Application Data
(60) Provisional application No. 63/656,249, filed on Jun. 5, 2024.

(51) Int. Cl. A61F 13/511; D04H 1/02; D04H 3/00

(52) U.S. Cl. CPC A61F 13/5116; D04H 1/02; D04H 3/00; A61F 2013/51182

(57) ABSTRACT

An air through bonded nonwoven material is provided. The air-through bonded nonwoven material comprises a plurality of fibers, a first plurality of areas formed in the fibers and having a first fiber to fiber bond density, and a second plurality of areas formed in the fibers and having a second, different fiber-to-fiber bond density. The first plurality of areas do not overlap with the second plurality of areas. The first fiber to fiber bond density is greater than the second fiber to fiber bond density. The first plurality of areas are substantially free of a film. The second plurality of areas are substantially free of a film.

### CROSS REFERENCE TO RELATED APPLICATION

This application claims the benefit, under 35 U.S.C. § 119 (e), of U.S. Provisional Patent Application No. 63/656,249, filed on Jun. 5, 2024, the entire disclosure of which is incorporated herein by reference.

### FIELD

The present disclosure is directed, in part, to nonwoven materials, and more specifically to nonwoven materials with variable fiber to fiber bond densities across their area.

### BACKGROUND

Nonwoven materials are used in many products. One example product is absorbent articles, such as diapers, pants, feminine hygiene products, and adult incontinence products, for example. Another example product is wet or dry wipes used for many purposes, such as cleaning portions of the body or cleaning surfaces. Typical nonwoven materials are formed by depositing fibers on a collection surface and then point, calendar, or air though bonding the fibers together to obtain an integral web that has integrity (i.e., can be handled without the fibers falling apart). Once the web is formed and has bonding throughout, other processes may be used to create structure in the nonwoven materials, such as three-dimensional elements, densified areas, and/or apertures, for example. During these processes, however, fiber movement is restricted because the bonds already formed in the nonwoven materials hold the fibers in place and restrict their movement. Moreover, such processes can potentially damage the fiber bonds in nonwovens and make the created structures weaker. This restriction of movement of the fibers creates structures in the nonwoven that are not desirable in strength, formation, and/or compression resistance. As such, nonwoven materials and processes for producing the same should be improved.

### SUMMARY

The present disclosure is directed, in part, to nonwoven materials that have a plurality of areas of lower fiber to fiber bond density and a plurality of areas of higher fiber to fiber bond density. The areas of higher fiber to fiber bond density may be locally densified areas, aperture rings surrounding apertures, and/or three-dimensional elements. To create such structures, fibers are laid down on a collection surface (e.g., belt or drum) and then structure (e.g., local densification, three-dimensional element formation, aperturing) is created in a way that an increased fiber to fiber contact density is achieved without bonding or with very minimal bonding present in the collected fibers. The fibers are then air through, or otherwise, bonded to lock the fibers in the structures and non-structured areas in place. The air through bonding creates fiber to fiber bonds where the fibers intersect or touch each other in the nonwoven materials. Owing to the fact that the fibers are moved into certain structures prior to any bonding taking place, allows areas that have higher fiber to fiber bond densities and areas that have lower fiber to fiber bond densities to be created. Examples of areas having higher bond densities are localized densified regions, three-dimensional features, and/or aperture rings surrounding apertures. The structures may be created by embossing, aperturing, and/or other tooling to create three-dimensional elements.

The benefit of moving the fibers or increasing their contact with each other prior to any bonding (e.g., point, calendar, air through bonding) is achieving higher fiber to fiber bond densities in the structures providing added strength, compression resistance, and/or better formation. For example, an aperture ring having high fiber to fiber bond densities forms better/cleaner apertures in the nonwoven material because of the additional bonding in that area, moreover such apertures are more stable under compression. As another example, a three-dimensional element with higher fiber to fiber bond densities can resist compression and/or collapse due to the strength added by all the fiber to fiber bonds.

The present disclosure is directed, in part, to an air through bonded nonwoven material comprising a plurality of fibers, a first plurality of areas formed in the fibers and having a first fiber to fiber bond density, and a second plurality of areas formed in the fibers and having a second fiber to fiber bond density. The first plurality of areas do not overlap with the second plurality of areas. The first fiber to fiber bond density is greater than the second fiber to fiber bond density. The first plurality of areas are substantially free of a film. The second plurality of areas are substantially free of a film.

The present disclosure is directed, in part, to an air through bonded nonwoven material comprising a plurality of fibers, a plurality of apertures defined in the plurality of fibers, aperture rings surrounding the apertures, and a land areas surrounding the aperture rings. The aperture rings have a first fiber to fiber bond density. The land areas have a second fiber to fiber bond density. The first fiber to fiber bond density is greater than the second fiber to fiber bond density.

The present disclosure is directed, in part, to a method of producing a nonwoven material comprising conveying unconsolidated fibers, creating locally densified areas or apertures comprising aperture rings in the unconsolidated fibers, air through bonding the nonwoven material to create a greater fiber to fiber bond density in the locally densified areas or aperture rings compared to a fiber to fiber bond density in areas without the locally densified areas or the apertures comprising aperture rings, and winding or slitting the nonwoven material. The first plurality of areas are substantially free of a film. The second plurality of areas are substantially free of a film.

### BRIEF DESCRIPTION OF FIGURES

While the specification concludes with claims particularly pointing out and distinctly claiming the subject matter which is regarded as forming the present invention, it is believed that the invention will be better understood from the following description which is taken in conjunction with the accompanying drawings in which the designations are used to designate substantially identical elements and in which:

FIG. 1 is a flow diagram illustrating how conventional non-variable fiber to fiber bond nonwoven materials are produced;

FIG. 2 is a flow diagram illustrating how variable fiber to fiber bond nonwoven materials of the present disclosure are produced;

FIG. 3 is an example of air through bonds 36 at fiber intersections in the nonwoven materials of the present disclosure;

FIG. 4 is an example of higher fiber to fiber bond densities around an aperture ring compared to areas distal from the aperture ring;

FIG. 5 illustrates apertures in a nonwoven material formed by the process of FIG. 1;

FIG. 6 illustrates apertures in a nonwoven material formed by the process of FIG. 2;

FIG. 7 illustrates the performance of the apertures in a nonwoven material formed by the process of FIG. 1 under compressive load;

FIG. 8 illustrates the performance of the apertures in a nonwoven material formed by the process of FIG. 2 under compressive load;

FIG. 9 illustrates a nonwoven material formed by the process of FIG. 2 with high fiber to fiber bond densities in the aperture rings compared to the fiber to fiber bond densities in the land areas;

FIG. 10 illustrates an example of an apertured nonwoven material;

FIG. 10A illustrates densification in a nonwoven material formed by the process in FIG. 1;

FIG. 10B illustrates densification in a nonwoven material formed by the process in FIG. 2;

FIG. 11 illustrates an example of a locally densified nonwoven material;

FIG. 12 is an example of a portion of a nonwoven material comprising a plurality of fibers defining densification;

FIG. 12A is an example of a portion of a nonwoven material comprising a plurality of fibers with a film where indicated;

FIG. 13 an example of a portion of a nonwoven material comprising a plurality of fibers defining an aperture;

FIG. 14 is an example nonwoven material made using the process of FIG. 2 illustrating apertures and three-dimensional features;

FIG. 15 is an example of a portion of an apertured nonwoven materials for the process in FIG. 1 with bonded areas of interest labelled;

FIG. 16 is an example of a portion of an apertured nonwoven materials for the present disclosure with bonded areas of interest labelled;

FIG. 17 is an example of a portion of a densified nonwoven materials for the process in FIG. 1 with bonded areas of interest labelled;

FIG. 18 is an example of a portion of a densified nonwoven materials for the present disclosure with bonded areas of interest labelled;

FIG. 19 illustrates the method for determining fiber to fiber bonding; and

FIG. 20 illustrates the method for determining film in the nonwoven.

### DETAIL DESCRIPTION

Various non-limiting forms of the present disclosure will now be described to provide an overall understanding of the principles of the structure, function, manufacture, and use of the nonwoven materials with variable fiber to fiber bond density disclosed herein. One or more examples of these non-limiting forms are illustrated in the accompanying drawings. Those of ordinary skill in the art will understand that the nonwoven materials with variable fiber to fiber bond density specifically described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

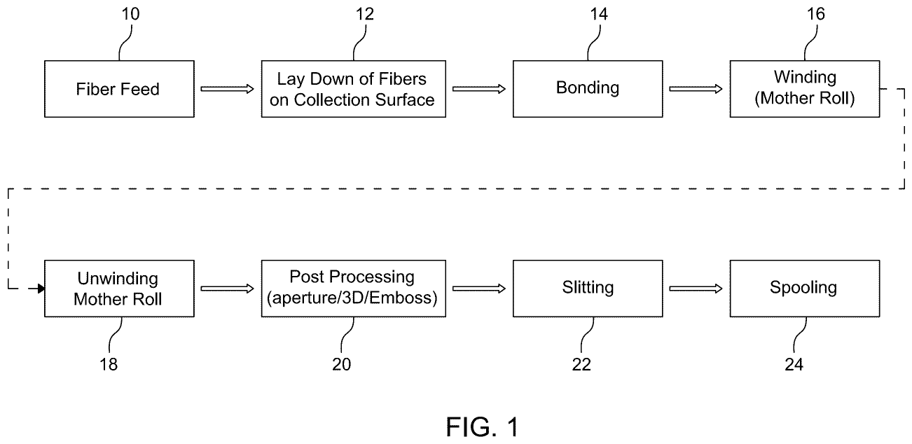

Referring to FIG. 1, nonwoven materials are typically formed by feeding fibers 10 to a collection surface, laying down the fibers on the collection surface 12 (e.g., belt or drum) to form an unconsolidated web, and performing some type of bonding 14 to enable the web to have self-sustaining integrity. Without such bonding, the web would merely be a loose layer of fibers. The web is then wound 16 into a mother roll. The is the typical process for forming flat nonwoven materials, whether carded, spunbond, or otherwise. Any post processing (e.g., aperturing, local densification, or three-dimensional element formation) typically occurs after the web is initially formed and is bonded to have integrity so it can be wound. Starting with a web that is already bonded, however, prevents, or at least inhibits, fiber movement or increasing fiber to fiber contact in certain areas during post processing. The fibers in the web are essentially locked in place by the bonding and are limited in the movement or increase in fiber to fiber contact they can achieve during post processing. Moreover, such post bonding processes weaken the existing bonds in the nonwoven material and make the created structures weaker.

In a typical post processing step, the mother roll of the flat nonwoven material is unwound 18, the post processing occurs 20, the nonwoven material is slit 22 into appropriate widths, for example for a topsheet for an absorbent article, and then the nonwoven materials is wound again into spools 24. The wound spools of the nonwoven materials have generally uniform fiber to fiber bond densities throughout their area owing to the fact that the nonwoven materials were bonded uniformly prior to any post processing or fiber movement taking place. This can lead to weaker or non-ideal formation of three-dimensional elements, apertures, and/or embossments.

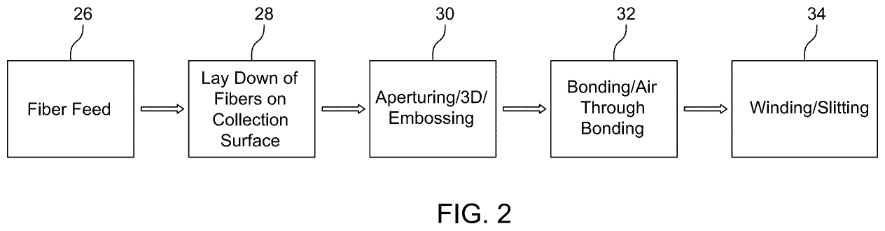

The present inventors have discovered that while more fiber movement can be achieved by performing the three-dimensional element, aperture, or local densification/embossment formation prior to initial bonding of the fibers of the web, it can also be done in a way to create local higher fiber to fiber bond density areas to stabilize these structures better. Referring to FIG. 2, a plurality of fibers may be fed 26 to a collection surface where they may be laid down 28. Prior to any bonding occurring or minimal bonding occurring (i.e., fibers are not joined or adequately joined to each other), the loose web of fibers may then be apertured, locally densified/embossed, or have three-dimensional elements formed therein 30 in a way to create specific areas with higher fiber to fiber contact population per mass. The formed loose fibers may then be bonded or air through bonded 32 to essentially set or lock in place the aperture rings, local densified areas/embossments, and/or three-dimensional features and also provide the web with integrity. The nonwoven material may then be slit and wound 34.

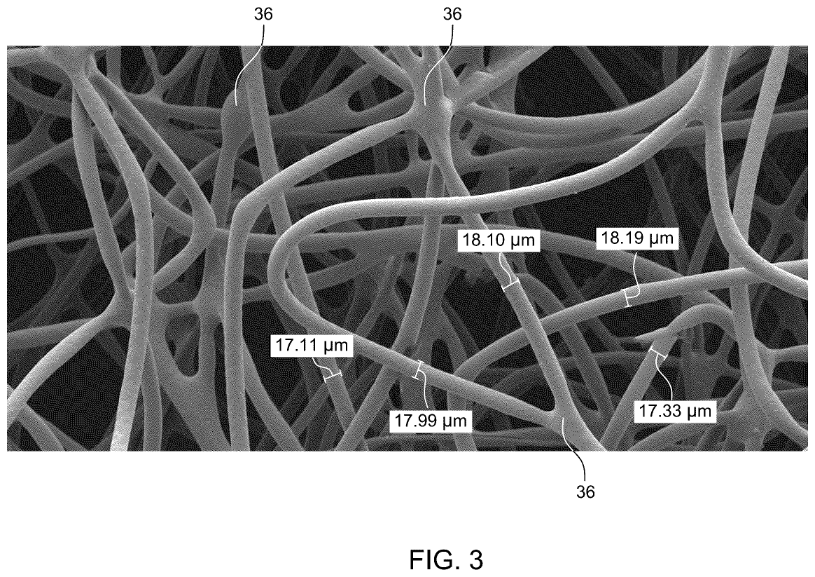

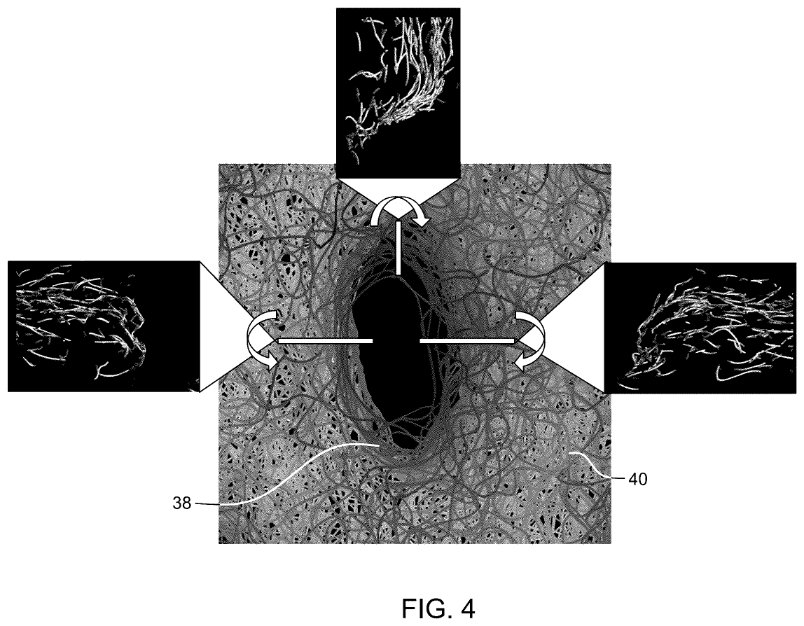

Owing to the fact that the fibers were moved or altered during aperturing, local densification/embossing, and/or three-dimensional element formation in a way to make more fibers have more contact with each other in certain areas of the nonwoven material, such as in aperture rings (including aperture tails) or locally densified areas, the formed structures have more integrity. In an air through bonding context, more fiber to fiber bonds will naturally occur where more fibers are contacting each other. Air through bonding is pushing a hot fluid through the web, thereby melting fiber to fiber intersections. An example of air through bonds 36 at fiber to fiber intersections is illustrated in FIG. 3. Referring to FIG. 4, there may be a higher density of fiber to fiber bonds where more fiber to fiber intersections are present, such as in aperture rings 38, compared to lower fiber to fiber bond areas 40 or land areas of the nonwoven material outside the aperture rings. It can be seen in FIG. 4 how a denser population of fiber to fiber intersections exist around the aperture compared to areas further from the aperture. The more fiber to fiber intersections in the aperture ring leads to more fiber to fiber bonds when the nonwoven material is air through bonded. This leads to better aperture formation and stability. Any of the apertures discussed herein may be formed using hydroentangling or fluid jets to create the apertures comprising aperture rings and optionally aperture tails.

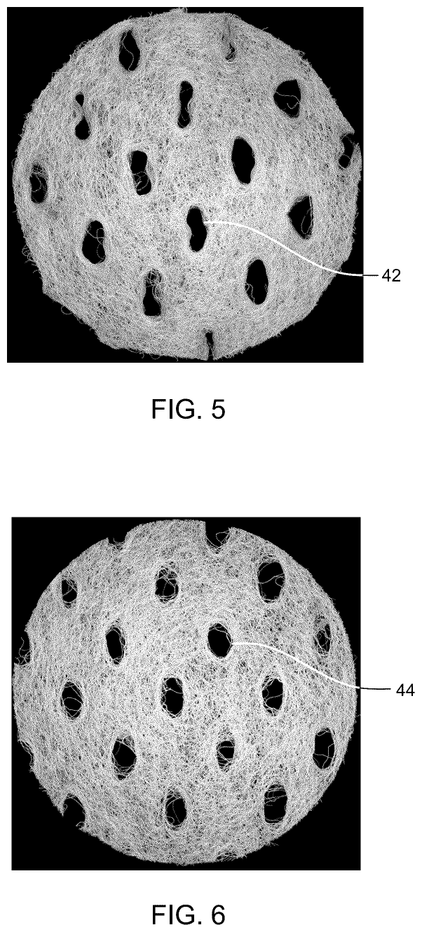

For further illustration, FIG. 5 illustrates apertures 42 formed by the process of FIG. 1 while FIG. 6 illustrates apertures 44 formed by the process of FIG. 2. Clearly, the apertures 44 of FIG. 6 have better formation and stability owing to the higher fiber to fiber bond density in the aperture rings compared to the apertures 42 of FIG. 5.

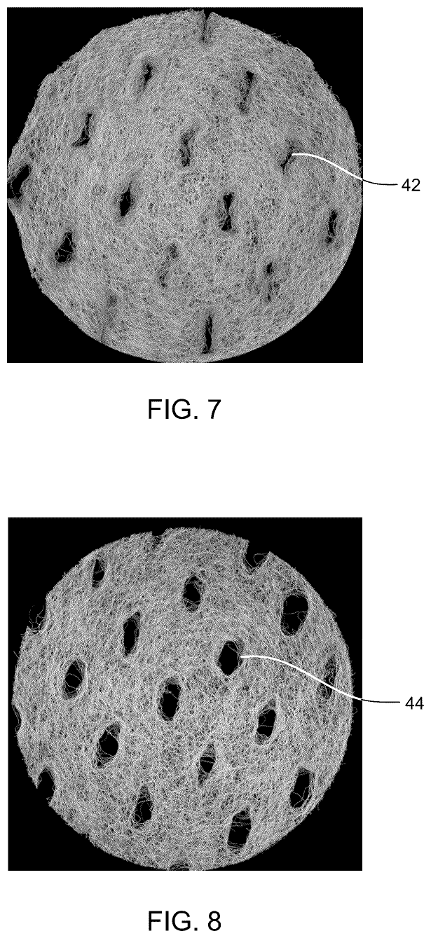

For still further illustration, FIG. 7 illustrates apertures 42 formed by the process of FIG. 1 while FIG. 8 illustrates apertures 44 formed by the process of FIG. 2. In this case, an identical compressive load was added to both nonwovens. Clearly, the apertures 44 of FIG. 8 maintain their shape owing to the higher fiber to fiber bond density in the aperture rings compared to the misshaped apertures 42 of FIG. 7.

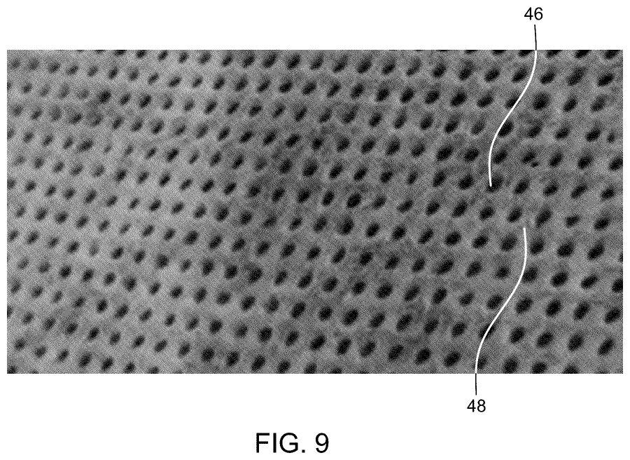

FIG. 9 illustrates an apertured nonwoven material formed by the process of FIG. 2 with high fiber to fiber bond densities in the aperture rings 46 (including aperture tails, if any) compared to the fiber to fiber bond densities in the land areas 48. It can be seen how clean the apertures have been formed.

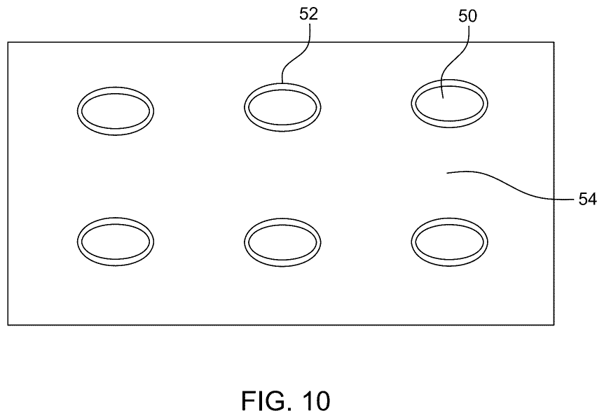

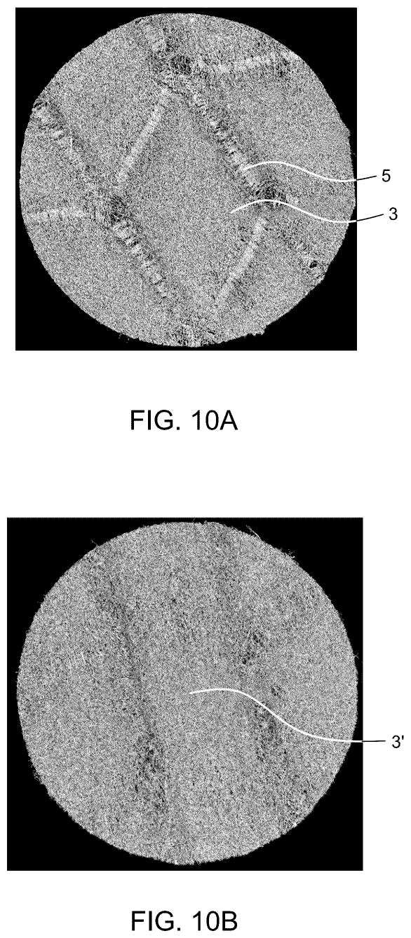

FIG. 10 illustrates an example of an apertured nonwoven material of the present disclosure. The nonwoven material comprises apertures 50 having aperture rings 52 (that may include aperture tails) and land areas 54 surrounding the aperture rings 52. The aperture rings 52 may have higher fiber to fiber bond densities than the land areas 54 owning to the process discussed with respect to FIG. 2. More fiber to fiber intersections exist in the aperture rings 52 than in the land areas 54.

Also, there may be a higher density of fiber to fiber bond density in embossments where the fibers are more compressed (causing more fiber to fiber contact) compared to areas of the nonwoven material outside of the embossments (less fiber to fiber contact). There also may be a higher density of fiber to fiber bonds in at least portions of three-dimensional elements where more fiber to fiber intersections exist compared to areas of the nonwoven material outside the three-dimensional elements (less fiber to fiber contact).

FIG. 10A illustrates densification 3 formed by the process of FIG. 1 while FIG. 10Y illustrates densification 3′ formed by the process of FIG. 2. Clearly, the densified 44 of FIG. 10X shows compaction of fibers in the embossment channel. In many instances, the channel forms a solidified surface or film in the embossment. Film can impact softness of the nonwoven without improving stability. Upon information and belief, FIG. 10B stability is owing to the non-film fiber to fiber bond density which is slightly greater than FIG. 10A in the embossments. Film is indicated as 5 in FIG. 10A.

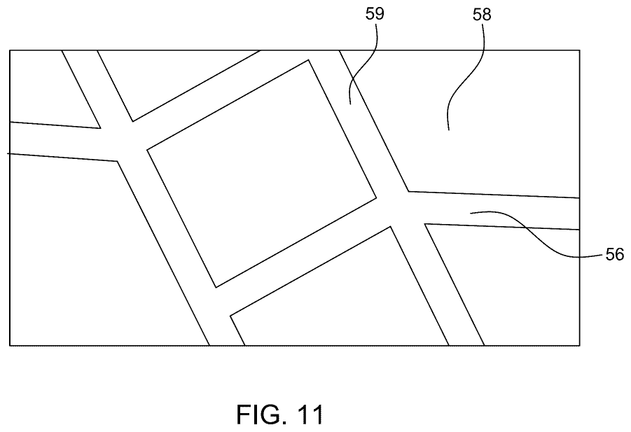

FIG. 11 illustrates an example of a locally densified nonwoven material of the present disclosure. The nonwoven material comprises locally densified areas 56, such as embossments, for example, and land areas 58 surrounding the locally densified areas 56. The locally densified areas 56 do not overlap with the land areas 58. Both the locally densified areas 56 and the land areas 58 are free of, or substantially free of a film formed due to fused or molten fibers. The locally densified areas 56 have higher fiber to fiber bond densities than the land areas 58 owing to the process discussed with respect to FIG. 2. More fiber to fiber intersections exist in the locally densified areas 56 than in the land areas 58. Film may not occur in area 59.

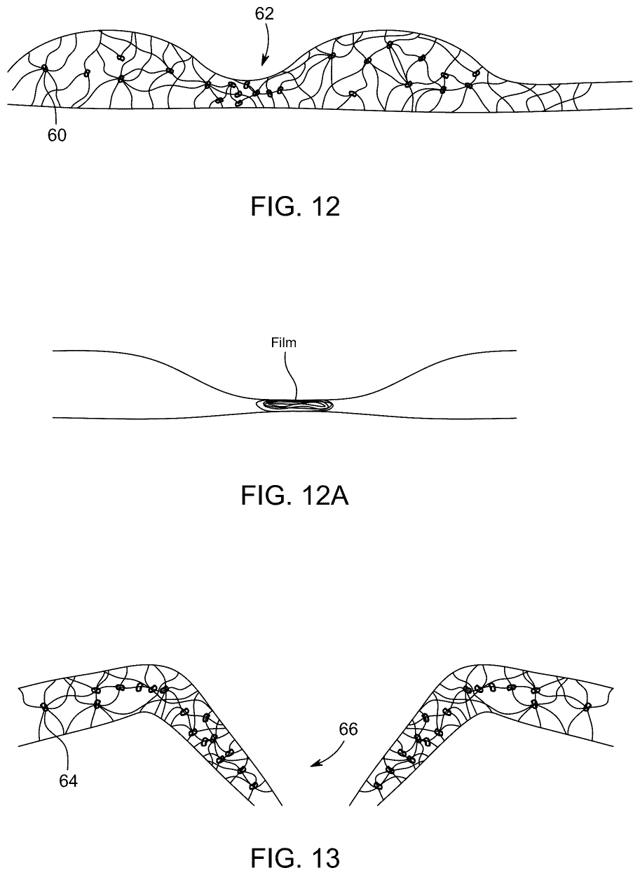

FIG. 12 an example of a portion of a nonwoven material comprising a plurality of fibers. The plurality of fibers comprises a first area formed in the plurality of fibers having a first, higher fiber to fiber bond densities in a locally densified region 60 and a second area formed in the plurality of fibers having a second, lower fiber to fiber bond densities in a non-densified region 62. This type of structure is creatable due to the process discussed with respect to FIG. 2. More fiber to fiber intersections are created in the locally densified region 60 than in the second non-densifies region 62. A ratio of the first, higher fiber to fiber bond density to the second, lower, fiber to fiber bond density is the range of about 0.1 to about 10, about 0.5 to about 10, about 0.75 to about 8, about 1.0 to about 7.0, about 1.0 to about 6.0, about 1.0 to about 5.0, about 1.0 to about 4.0, about 1.0 to about 3.0, about 1.5 to about 2.5, about 2.0, or about 2.1, for example. FIG. 12A is an example of a portion of a nonwoven material comprising a plurality of fibers with a film where indicated.

FIG. 13 an example of a portion of a nonwoven material comprising a plurality of fibers defining an aperture. The plurality of fibers comprise first, higher fiber to fiber bond densities in an aperture ring and tail 64 and second, lower fiber to fiber bond densities in non-apertured regions 66. This type of structure is creatable due to the process discussed with respect to FIG. 2. More fiber to fiber intersections are created in the aperture ring and tail 64 than in the second non-apertured regions 66. A ratio of the first, higher fiber to fiber bond density to the second, lower, fiber to fiber bond density is the range of about 0.1 to about 10.0, about 0.5 to about 8.0, about 0.5 to about 7.0, about 0.5 to about 6.0, about 0.5 to about 5.0, about 0.5 to about 4.0, about 0.5 to about 3.0, about 0.5 to about 2.0, about 0.75 to about 1.75, about 0.75 to about 1.5, about 0.9, about 1.0, about 1.1, about 1.2, about 1.3, or about 1.4, for example.

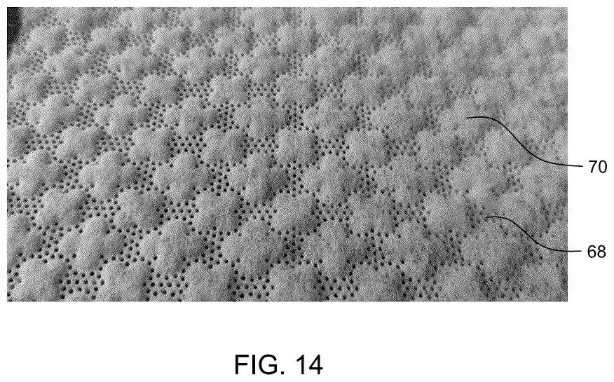

FIG. 14 is an example nonwoven material made using the process of FIG. 2 illustrating apertures 68 and three-dimensional features 70. Aperture rings (and optionally aperture tails) surrounding the apertures may have a higher fiber to fiber bond density than the areas forming the three-dimensional features.

The fibers used in the process of the present disclosure and the nonwoven materials themselves may comprise PE/PP, PE/PET, or PP/PP bicomponent fibers.

The fibers may comprise any suitable thermoplastic polymers. Example thermoplastic polymers are polymers that melt and then, upon cooling, crystallize or harden, but that may be re-melted upon further heating. Suitable thermoplastic polymers may have a melting temperature (also referred to as solidification temperature) from about 60° C. to about 300° C., from about 80° C. to about 250° C., or from about 100° C. to about 215° C. And, the molecular weight of the thermoplastic polymer may be sufficiently high to enable entanglement between polymer molecules and yet low enough to be melt spinnable.

The thermoplastic polymers may be derived from any suitable material including renewable resources (including bio-based and recycled materials), fossil minerals and oils, and/or biodegradeable materials. Some suitable examples of thermoplastic polymers include polyolefins, polyesters, polyamides, copolymers thereof, and combinations thereof. Some example polyolefins include polyethylene or copolymers thereof, including low density, high density, linear low density, or ultra-low density polyethylenes such that the polyethylene density ranges between about 0.90 grams per cubic centimeter to about 0.97 grams per cubic centimeter or between about 0.92 and about 0.95 grams per cubic centimeter, for example.

The thermoplastic polymer component may be a single polymer species or a blend of two or more thermoplastic polymers e.g., two different polypropylene resins. As an example, fibers of a first layer of the nonwoven material may comprise polymers such as polypropylene and blends of polypropylene and polyethylene, while a second layer of the nonwoven material may comprise fibers selected from polypropylene, polypropylene/polyethylene blends, and polyethylene/polyethylene terephthalate blends. In some forms, one of the layers of the nonwoven material may comprise fibers selected from cellulose rayon, cotton, other hydrophilic fiber materials, or combinations thereof. The fibers may also comprise a super absorbent material such as polyacrylate or any combination of suitable materials.

The fibers may comprise monocomponent fibers, bi-component fibers, and/or bi-constituent fibers, round fibers or non-round fibers (e.g., capillary channel fibers), and may have major cross-sectional dimensions (e.g., diameter for round fibers) ranging from about 0.1 microns to about 500 microns. The fibers may also be a mixture of different fiber types, differing in such features as chemistry (e.g. polyethylene and polypropylene), components (mono- and bi-), denier (micro denier and >2 denier), shape (i.e. capillary and round) and the like. The fibers may range from about 0.1 denier to about 100 denier.

Example nonwoven materials are contemplated where a first plurality of fibers and/or a second plurality of fibers comprise additives in addition to their constituent chemistry. For example, suitable additives include additives for coloration, antistatic properties, lubrication, softness, hydrophilicity, hydrophobicity, and the like, and combinations thereof. These additives, for example titanium dioxide for coloration, may generally be present in an amount less than about 5 weight percent and more typically less than about 2 weight percent or less.

As used herein, the term “monocomponent fiber(s)” refers to a fiber formed from one extruder using one or more polymers. This is not meant to exclude fibers formed from one polymer to which small amounts of additives have been added for coloration, antistatic properties, lubrication, hydrophilicity, etc.

As used herein, the term “bi-component fiber(s)” refers to fibers which have been formed from at least two different polymers extruded from separate extruders but spun together to form one fiber. Bi-component fibers are also sometimes referred to as conjugate fibers or multicomponent fibers. The polymers are arranged in substantially constantly positioned distinct zones across the cross-section of the bi-component fibers and extend continuously along the length of the bi-component fibers. The configuration of such a bi-component fiber may be, for example, a sheath/core arrangement wherein one polymer is surrounded by another, or may be a side-by-side arrangement, eccentric, a pie arrangement, or an “islands-in-the-sea” arrangement. Some specific examples of fibers which may be used in the first nonwoven layer include polyethylene/polypropylene side-by-side bi-component fibers. Another example is a polypropylene/polyethylene bi-component fiber where the polyethylene is configured as a sheath and the polypropylene is configured as a core within the sheath. Still another example is a polypropylene/polypropylene bi-component fiber where two different propylene polymers are configured in a side-by-side configuration. Additionally, forms are contemplated where the fibers of a nonwoven layer are crimped.

Bi-component fibers may comprise two different resins, e.g. a first polypropylene resin and a second polypropylene resin. The resins may have different melt flow rates, molecular weights, or molecular weight distributions. Ratios of the 2 different polymers may be about 50/50, 60/40, 70/30, 80/20, or any ratio within these ratios. The ratio may be selected to control the amount of crimp, strength of the nonwoven layer, softness, bonding or, the like.

As used herein, the term “bi-constituent fiber(s)” refers to fibers which have been formed from at least two polymers extruded from the same extruder as a blend. Bi-constituent fibers do not have the various polymer components arranged in relatively constantly positioned distinct zones across the cross-sectional area of the fiber and the various polymers are usually not continuous along the entire length of the fiber, instead usually forming fibrils which start and end at random. Bi-constituent fibers are sometimes also referred to as multi-constituent fibers. In other examples, a bi-component fiber may comprise multiconstituent components.

As used herein, the term “non-round fiber(s)” describes fibers having a non-round cross-section, and includes “shaped fibers” and “capillary channel fibers.” Such fibers may be solid or hollow, and they may be tri-lobal, delta-shaped, and may be fibers having capillary channels on their outer surfaces. The capillary channels may be of various cross-sectional shapes such as “U-shaped”, “H-shaped”, “C-shaped” and “V-shaped”. One practical capillary channel fiber is T-401, designated as 4DG fiber available from Fiber Innovation Technologies, Johnson City, TN. T-401 fiber is a polyethylene terephthalate (PET polyester).

Other example nonwoven materials may comprise spunbond materials, carded materials, melt blown materials, spunlace materials, needle punched materials, wet-laid materials, or air-laid materials, for example.

The nonwoven webs of the present disclosure may have one or more layers. The one or more layers may have the same or different fiber types. The fibers in each layer may have the same or different deniers. The layers may have the same or different surface energy.

The nonwoven webs of the present disclosure may have variable intensive properties throughout their areas. Example nonwoven webs having variable intensive properties are disclosed in U.S. Pat. No. 10,888,471. When the variable intensive property nonwoven material is laid down on the collection surface, no bonding or very little bonding may occur until aperturing, local densification, and or three-dimensional element formation occurs. The intensive properties may be basis weight, volumetric density, and/or thickness. As an example, a nonwoven web may have first regions with a first basis weight and second regions with a second basis weight. The first basis weight may be lower than the second basis weight. The first regions, however, may have higher fiber to fiber intersections and thereby fiber to fiber bonds than the second regions. The first regions may have a higher density than the second regions.

### Method

A method of producing a nonwoven material having variable densities of fiber to fiber bonds may comprise conveying unconsolidated fibers, creating local densified areas with increased fiber to fiber contact, or creating apertures comprising aperture rings with increased fiber to fiber contact in the unconsolidated fibers. The method may comprise air through bonding the nonwoven material to create a greater fiber to fiber bond intersection densities in the locally densified areas or in the aperture rings compared to a fiber to fiber bond intersection densities in areas without the locally densified areas or the apertures comprising aperture rings, and winding or slitting the nonwoven material. A ratio of the fiber to fiber bond intersection densities in the locally densified areas or aperture rings to the fiber to fiber bond intersection densities outside the locally densified areas or aperture rings may be about 1.1 to about 10, about 1.2 to about 10, about 1.3 to about 10, about 1.4 to about 10, about 1.5 to about 10, about 1.6 to about 10, about 1.7 to about 10, about 1.8 to about 10, about 1.9 to about 10, about 2 to about 10, about 3 to about 10, about 4 to about 10, about 5 to about 10, or about 6 to about 10. The unconsolidated fibers may comprise continuous spun fibers or carded fibers. The aperture rings may have a higher basis weight compared to areas without the aperture rings. The method may comprise using fluid jets to create the apertures comprising aperture rings.

TABLE 1

| | | | | | Bond |
|---|---|---|---|---|---|
| | | Average | | | remove |
| | ROI | Basis | Bond | Film | Film |
| | Area | Weight | Volume | Volume | Volume |
| | (mm\hat{ }2) | (gsm) | (%) | (%) | (%) |
| Conventional Method Land1 | 4.6 | 37.7 | 21.5 | 0.5 | 21 |
| Conventional Method Land2 | 3.2 | 49.3 | 22 | 0.7 | 21.3 |
| Conventional Method Land3 | 4.5 | 43.5 | 22.3 | 0.8 | 21.5 |
| Conventional Method Land4 | 4.5 | 43.1 | 22.2 | 0.6 | 21.6 |
| TOTAL: | 16.8 | | | | |
| AVERAGE: | | 43.4 | | | 21.4 |
| Conventional Method Aperture1 | 6.6 | 43.1 | 21.2 | 0.8 | 20.5 |
| Conventional Method Aperture2 | 5 | 38.8 | 19.9 | 0.7 | 19.3 |
| Conventional Method Aperture3 | 7.1 | 45.3 | 23.3 | 1.1 | 22.5 |
| Conventional Method Aperture4 | 7.8 | 39.7 | 22.6 | 0.8 | 21.9 |
| TOTAL: | 26.5 | | | | |
| AVERAGE: | | 41.7 | | | 21.1 |

RATIO OF BOND VOLUMES: 1.0

| | | | | | |
|---|---|---|---|---|---|
| Disclosed Method Land1 | 6 | 38.6 | 30.9 | 1.2 | 29.7 |
| Disclosed Method Land2 | 6 | 31.3 | 27.9 | 0.8 | 27 |
| Disclosed Method Land3 | 4.8 | 37.6 | 29.5 | 1.1 | 28.4 |
| Disclosed Method Land4 | 4.7 | 34 | 26.8 | 0.9 | 25.9 |
| Disclosed Method Land5 | 4.5 | 34.4 | 31 | 1.2 | 29.8 |
| TOTAL: | 26.0 | | | | |
| AVERAGE: | | 35.2 | | | 28.2 |
| Disclosed Method Aperture1 | 3.5 | 34.9 | 42.1 | 2.1 | 40 |
| Disclosed Method Aperture2 | 4.3 | 36.7 | 40.3 | 2 | 38.4 |
| Disclosed Method Aperture3 | 4.4 | 33.6 | 40.1 | 2 | 38.1 |
| Disclosed Method Aperture4 | 3.8 | 28.8 | 37.8 | 1.2 | 36.7 |
| TOTAL: | 16 | | | | |
| AVERAGE: | | 33.5 | | | 38.3 |

RATIO OF BOND VOLUMES: 1.4

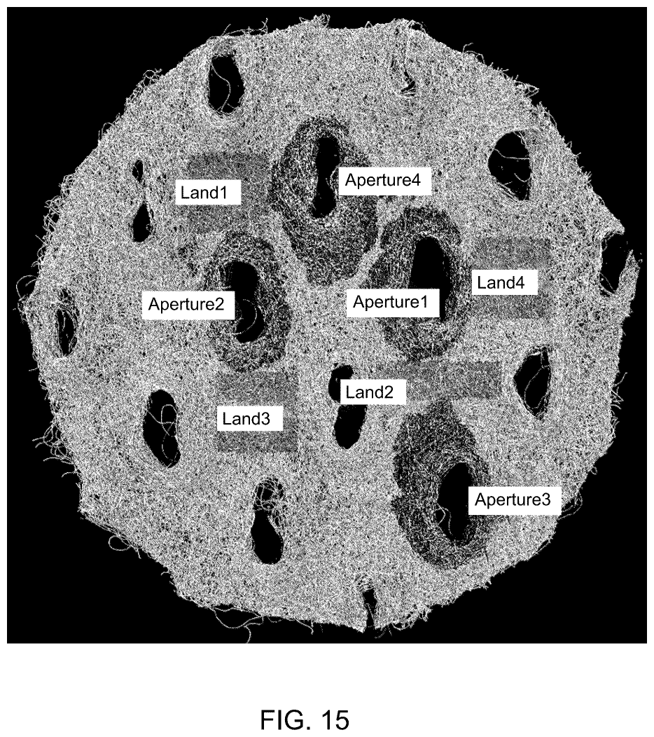

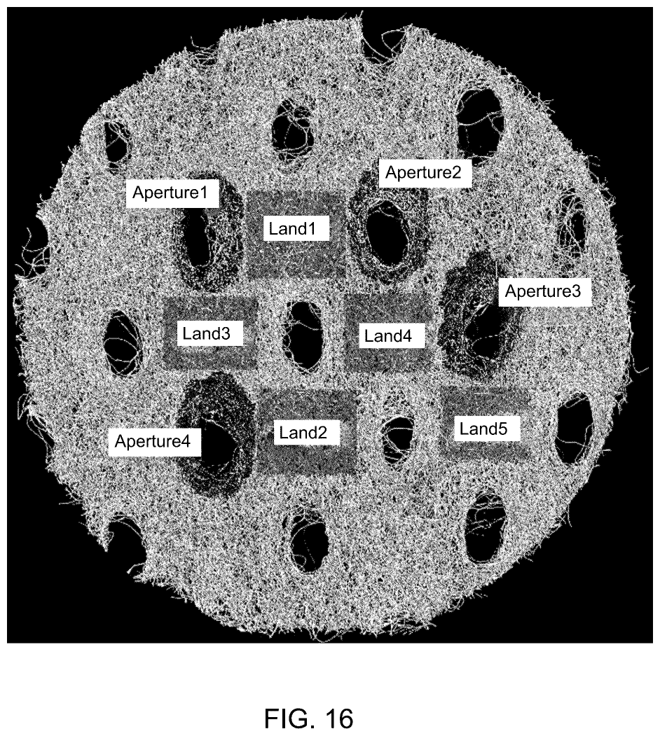

Referring to FIG. 15. the nonwoven material of the comparative example is a spunbond nonwoven with a 26 gsm basis weight. This material was apertured (HOW) after it was through air bonded. As can be seen above. the fiber to fiber bond points in the aperture is significantly higher than the fiber to fiber bond points in the land areas.

TABLE 2

| | | | | | Bond |
|---|---|---|---|---|---|
| | | Average | | | remove |
| | ROI | Basis | Bond | Film | Film |
| | Area | Weight | Volume | Volume | Volume |
| | (mm\hat{ }2) | (gsm) | (%) | (%) | (%) |
| Conventional Method Land1 | 14.1 | 43.6 | 12.2 | 0.1 | 12.2 |
| Conventional Method Land2 | 11.7 | 39.5 | 13.4 | 0.1 | 13.3 |
| TOTAL: | 25.8 | | | | |
| AVERAGE: | | 41.6 | | | 12.8 |
| Conventional Method Densify1 | 7.9 | 20.5 | 32.1 | 8.4 | 25 |
| Conventional Method Densify2 | 8.4 | 18.1 | 34.1 | 7.2 | 27.9 |
| Conventional Method Densify3 | 8.3 | 18 | 34.1 | 9.3 | 26.4 |
| Conventional Method Densify4 | 4.5 | 34.9 | 26.6 | 6.1 | 21.5 |
| TOTAL: | 29.1 | | | | |
| AVERAGE: | | 22.9 | | | 25.2 |

RATIO OF BOND VOLUMES: 2.0

| | | | | | |
|---|---|---|---|---|---|
| Disclosed Method Land1 | 14.1 | 51.5 | 15.2 | 0.1 | 15.1 |
| Disclosed Method Land2 | 14.1 | 47.1 | 18.8 | 0.2 | 18.6 |
| TOTAL: | 28.2 | | | | |
| AVERAGE: | | 49.3 | | | 16.9 |
| Disclosed Method Densify1 | 6.7 | 23.2 | 35.7 | 1.2 | 34.7 |
| Disclosed Method Densify2 | 5.1 | 23.4 | 39.1 | 1.5 | 37.8 |
| Disclosed Method Densify3 | 6.9 | 23.1 | 35.3 | 1.3 | 34.2 |
| Disclosed Method Densify4 | 5.3 | 32.2 | 36.2 | 1.3 | 35.1 |
| TOTAL: | 24 | | | | |
| AVERAGE: | | 25.5 | | | 35.5 |

RATIO OF BOND VOLUMES: 2.1

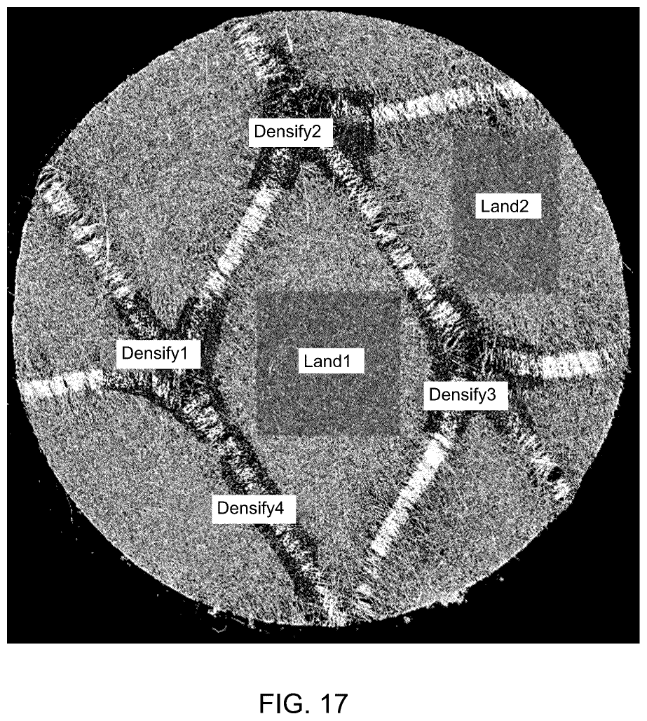

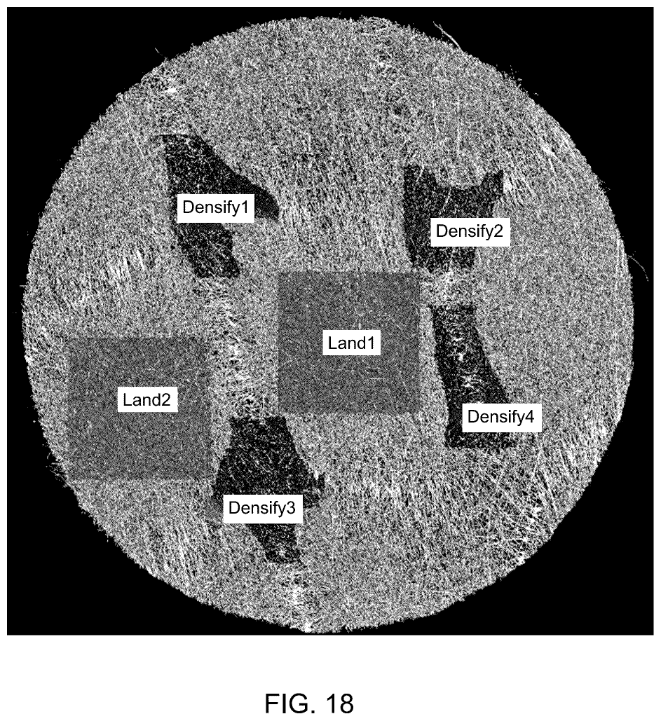

Referring to FIG. 17, the nonwoven material is a spunbond nonwoven with 30 gsm basis weight produced by the process shown in FIG. 1. Referring to FIG. 18, the nonwoven material of the present disclosure is a spunbond nonwoven with 28 gsm basis weight. The nonwoven material of FIG. 10B was made by laying down spunbond fibers on a textured drum and then subsequently air-through bonding the spunbond fibers to create the nonwoven web. As can been see in Table 2, this nonwoven material has higher fiber to fiber bond points between land and densified areas than FIG. 10A. FIG. 10B also formed the less desirable film in the embossments.

### Test Method

### Sample Preparation and Scanning

This micro-CT imaging and analysis method measures the bond points, basis weight, thickness, density values, and other parameters within visually discernible regions of a substrate sample. It is based on analysis of a 3D x-ray sample image obtained on a micro-CT instrument (a suitable instrument is the Scanco uCT 50 available from Scanco Medical AG, Switzerland, or equivalent). The micro-CT instrument is a cone beam micro-tomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and save the raw data. The 3D image can be visualized using visualization software (a suitable visualization software is Avizo available from Thermo Fisher Scientific Inc., Waltham, MA, or equivalent) and then analyzed using image analysis software (a suitable image analysis software is MATLAB available from The Mathworks, Inc., Natick, MA, or equivalent) to measure the desired properties of regions within the sample.

To obtain a sample for measurement, lay a single layer of the dry substrate material out flat and die cut a circular piece with a diameter of 16 mm. The sample weight should be recorded. A sample may be cut from any location containing the region to be analyzed. A region to be analyzed is one where there are visually discernible changes in texture, elevation, or thickness. Regions within different samples taken from the same substrate material can be analyzed and compared to each other. Care should be taken to avoid folds, wrinkles or tears when selecting a location for sampling.

Set up and calibrate the micro-CT instrument according to the manufacturer's specifications. Place the sample into the appropriate holder, stabilized between two rings of low-density material, which have an outer diameter of 16 mm and an inner diameter of 12 mm. The sample should be held planarly and aligned with the acquisition planes of the instrument.

The 3D image field of view is approximately 20 mm on each side in the XY-plane with a resolution of approximately 8192 by 8192 pixels, and with a sufficient number of 2.5 micron thick slices collected to fully include the Z-direction of the sample. The reconstructed 3D image contains isotropic voxels of 2.5 microns. Images were acquired with the source at 45 kVp and 88 uA with no additional low energy filter. These current and voltage settings should be optimized to produce the maximum contrast in the projection data with sufficient x-ray penetration through the sample, but once optimized held constant for all substantially similar samples. A total of 1500 projections images are obtained with an integration time of 400 ms and 4 averages. The projection images are reconstructed into the 3D image and saved in 16-bit format to preserve the full detector output signal for analysis.

Summarizing micro-CT data for Measurements

A threshold should be determined to separate fiber voxel from all other voxel in the micro-CT dataset. An automated technique such as Otsu method (implemented as the multithresh function in MATLAB) can be used to find the threshold. Connected components can be used to identify the largest volume in the dataset which will be the sampled nonwoven. All other volumes should be much smaller and can be removed as noise. The “Fiber Mask” resulting from the thresholding will assign fiber voxels a value of one.

Nonwoven fiber voxel intensities are proportional to fiber density. With the nonwoven flat and parallel to the XY plane as described in the sample preparation, a two-dimensional image can be generated by summing the fiber voxel values in the Z direction. This “projection image” is the sum of the fiber voxel densities as measured by the mCT at any XY position on the nonwoven. The area of the die cut nonwoven can be determined in this image with an outer hull algorithm such as the bwconvhull function available in MATLAB. The weight of the sample was measured prior to scanning and therefore the overall basis weight of the sample can be calculated using the determined area.

The “projection image” pixel density summation captured by the mCT can be related to basis weight. First determine the average “projection image” pixel value for the nonwoven area. Multiply the “projection image” by the ratio of true basis weight value divide by the average “projection image” pixel value to create the Basis Weight Image. Each pixel value in the Basis Weight Image approximates the basis weight for the area covered by that pixel. A continuous multi-pixels Region of Interest (ROI) within the Basis Weight Image gives a close approximation to the basis weight value for that area of the nonwoven by averaging the pixel values together. This is the method for determining mean basis weight of an ROI within the nonwoven.

With the nonwoven flat and parallel to the XY plane as described in the sample preparation and the punch or densifier/embosser moving in the minus Z direction, three additional two-dimensional images can be generated. The “Top Z Image” consists of the Z value of the first fiber voxel found at an XY location looking in the direction of the punch movement. The “Bottom Z Image” is the Z value of the last fiber voxel found at an XY location looking in the direction of the punch movement. The Z distance between the topmost fiber voxel and the bottommost fiber voxel will be referred to as the “Thickness Image”.

The pixel values of the “Basis Weight Image” are the basis weight based on the area of a pixel. The pixel value of the “Thickness Image” is the thickness of the nonwoven at a pixel. Dividing the “Basis Weight Image” by the “Thickness Image” on a pixel-by-pixel basis results in a volume in the denominator and creates a “Pixel Density Image”. The “Pixel Density Image” which is the density within a pixel volume can be used to find areas of high and low density within the nonwoven.

ROIs in a Nonwoven with Apertures

Comparing characteristics of ROIs in the aperture with land area ROIs will demonstrate the benefits of the invention. The aperture ROI may be decerned using 3D visualization. Punching a non-woven creates a hole through the nonwoven surrounded by an aperture ring of fibers that have been pushed towards the negative Z direction. As shown in FIG. 4, the ring may be asymmetrical due to the slackness of fibers during formation and handling after formation. For the samples shown in FIG. 5 and FIG. 6, the properties of the aperture ring begin at the edge of the aperture hole and expand into nonwoven by a distance equal to half the land area thickness. Half the average thickness should be sufficient to mask fibers affected by a punch which created walls averaging less than 25 degrees from the Z axis. The distance should be adjusted if the punching mechanism is designed to increase or decrease the wall angle. For the samples, the measurements could be automated using the “Top Z Image”. Thresholding the “Top Z Image” by an average of nonzero “Top Z Image” pixel values that surround the punch will locate the aperture near the middle of the aperture wall and will create an aperture mask. The mask is then dilated by half the average thickness of the land areas. Land area ROIs are continuous rectangular regions that are away from the effect of the punch. The “Top Z Image” analysis yielded four aperture ROIs and a similar number of land ROIs as shown in FIGS. 15 & 16.

ROIs in a Nonwoven with Densification

Comparing characteristics of ROIs in the higher density embossed areas versus lower density non-embossed areas will demonstrate the benefits of the invention. Determining the ROI of the embossed regions can be decerned using 3D visualization. Nonwovens formed by the process in FIG. 1 can contain highly compressed fibers in the embossment channel that result in film. The compression also alters the nearby fibers as indicate in FIG. 12. The size and shape of the alteration differ dependent on the formation process. For the samples shown in FIGS. 10X and 10Y, the measurements could be automated using the “Pixel Density Image”. The “Pixel Density Image” is confounded by pinholes (empty spaces in the nonwoven), stray fibers crossing above the channel and differing channel borders. Stray fibers are especially prevalent in material formed by the process in FIG. 2 and the borders were slightly bigger. A circular averaging filter with a diameter approximately equal to the channel width of an embossment sufficiently smoothed the image. Once the image has been filtered, an automated technique such as Otsu method (implemented as the multithresh function in MATLAB) can be used to find a threshold that separates the higher density areas from the lower density areas. Land area ROIs are continuous rectangular regions that are away from the effect of the embossment. The “Pixel Density Image” analysis yielded four densification ROIs and two land ROIs as shown in FIGS. 17 & 18.

### Measuring Fiber Bonds

Fiber bonding is when two or more fibers melt together. Bonding sites of the fibers will be determined with two morphological operators. The “Fiber Mask” is divided into datasets which have been trimmed to the X-Y boundaries of the previously identified ROIs. The morphological operators will set the values of fiber voxels based on morphological shapes. Only interior fiber voxels, voxels that are not in contact with empty space, are assigned values but the operators use all fiber voxels to determine the shape. The morphological operators are the Local Thickness Map (LTM) operator and the Local Bounding Box Map (LBBM) operator.

The LTM operator fits spheres into the interior of fibers. The LTM will assign to a fiber voxel value the diameter of the largest sphere that fits within the fiber and contains that voxel. For example, all voxel values within a spherical shaped object would be assigned the diameter of the sphere. This voxel value will be referred to as LTMDiam. This approach is documented in the article by R. P. Dougherty and K-H Kunzelmann, “Computing Local Thickness of 3D Structures with ImageJ,” Microscopy & Microanalysis, August 2007 with which is incorporated herein by reference. The method was implemented in MATLAB.

The Local Bounding Box Map (LBBM) operator fits the smallest arbitrarily oriented bounding box to a set of identified points on the surface of the fiber. The LBBM assigns three values which describes the box for each fiber voxel. The LBBM is computed by radiating vectors from a voxel. The vector directions are determined by a geodesic polyhedron which is a polyhedron that can approximate the shape of a sphere when enough triangles are specified. A simple example of a geodesic polyhedron is a Regular Octahedron which is composed of eight equilateral triangles surrounding the voxel. The octahedral used for the LBBM is described in the book Spherical Models, by Magnus Wenninger, Cambridge University Press, ISBN 978-0-521-29432-4, as a Class 1, m=4, n=0 octahedral which has 66 vertices and 128 triangles. This octahedral was chosen for its large density of nearly uniformly spaced direction vertices and because it has vertices falling on the positive and negative X, Y, Z axes. The vertices are used to defined vectors radiating from the interior fiber voxel to the first surface voxel encountered. This generates 66 points in space. If the vector does not reach a surface point within a maximum distance of 25 voxels, then the voxel point at the maximum distance is reported. Note that the 66 points may not be distinct if the interior voxel falls close to the fiber surface.

The 66 points are then used to find an Arbitrarily Oriented Minimum Bounding Box (AOMBB), which is described in the article by O'Rourke, Joseph. “Finding Minimal Enclosing Boxes.” International Journal of Computer and Information Sciences, vol. 14, no. 3, pp. 183-199, June 1985., incorporated herein by reference. This is the smallest box located anywhere in space, at any angle that will enclose all 66 points. The dimensions of a box are usually described by its length, width, and height. For AOMBB, the largest of these three values will be reported as the maximum distance (MaxBB), the smallest of these three values as the minimum distance (MinBB), and the remaining value as the middle-distance measure (MidBB). These three values MaxBB, MidBB, MinBB are assigned to each interior fiber voxel. The AOMBB algorithm used to compute these three values came from the MATLAB file exchange [Johannes Korsawe (2022). Minimal Bounding Box (https://www.mathworks.com/matlabcentral/fileexchange/18264-minimal-bounding-box), MATLAB Central File Exchange. Retrieved Feb. 22, 2022], incorporated herein by reference.

Fiber bonding occurs when two fibers are in contact, and their outer layers have melted together. Where two fibers are touching, their surface voxels become interior voxels that can allow an LBBM vector to pass through. Hence, LBBM can detect touching fibers. Fibers that have not absorbed enough heat to melt will have a nearly uniform diameter. When a fiber melts, the melt pools causes a larger diameter. The changes in diameter can be detected by the LTM. A fiber voxel is labelled part of a bond when the LTM detects a larger sphere which indicates a pool of melt and the LBBM detects a bounding box larger than the exterior of a single fiber which indicates a vector crossing to a touching fiber. Most of the voxels that fall within a bond volume will share both the larger diameter sphere and pass through bounding box.

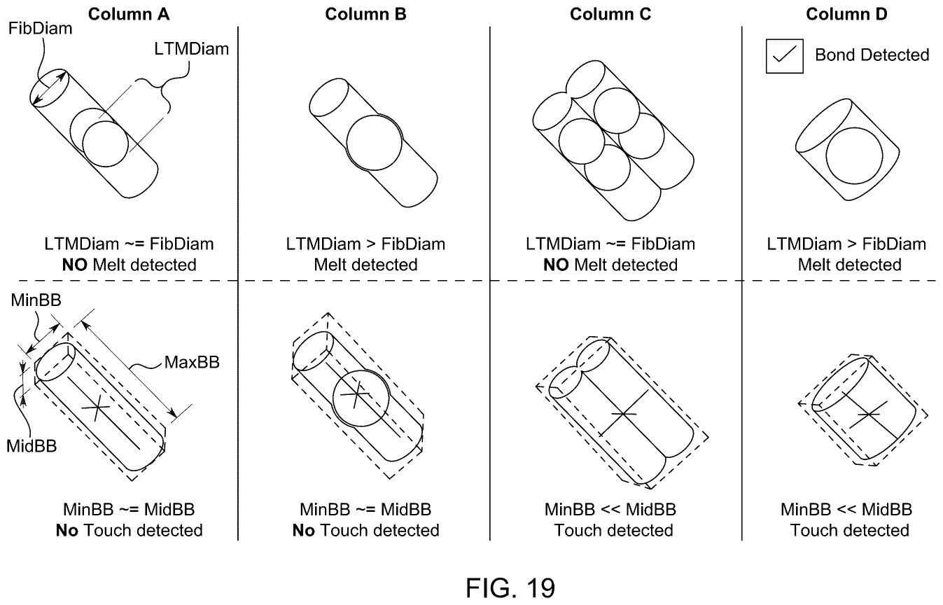

Columns A-D in FIG. 19 present simplified version of fiber geometry. Column A shows the case of a single fiber with the measurements defined above. In Column B, fiber melt occurs somewhere along the fiber which then appears as a drip of melt on the fiber with no bond. The case of melt with no bond appears in FIG. 3 at location XX. Column C shows two fibers that are simply touching with no melt creating a large bonding box. Finally, Column D is a section of fibers that have melted together and are identified by the two morphological operators as bonded.

Referring to Column A in the FIG. 19, if the fiber diameter (FibDiam) is nearly equal to the fitted sphere diameter (LTMDiam) then no melt is detected. Note that for melt to pool, sections of the fiber will lose mass and therefore the fiber diameter in the immediate vicinity may be smaller. Hence, the equation LTMDiam<FibDiam+CDIAM indicates no pooling melt and no bond. Likewise, the equation LTMDiam>FibDiam+CDIAM indicates melt. Epsilon eDIAM will be a delta that creates a value slightly greater than the average measured diameter of the fibers. One skilled in the art may adjust epsilon if the virgin fiber diameter is varying by an amount greater than the resolution of the scanner.

Referring to Column A, the maximum dimension (MaxBB) of a fitted box will normally follow the center path of the fiber. The fiber cross section will approximate a square enclosing the circular profile of the fiber. Hence, a MinBB is approximately equal to a MidBB. This can be codified in an equation MidBB<MinBB+eBB because the value of MidBB will always be greater than or equal to the value of MinBB. Where 2 fibers are touching, their surface voxels become interior voxels that can allow an LBBM vector to pass through. The equation MidBB>MinBB+eBB indicates touching fibers. Note that other fiber profiles may be codified with other length equations.

The bond percentage was determined from the LTM and LBBM voxel values. Each interior point of the fibers is assigned a true or false value based on the melting equation (LTMDiam>FibDiam+e_{DIAM}) and the touch equation (MidBB>MinBB+CBB) discussed above.

$$IsBondPoint = MidBB > MinBB + 2 \& LTMDiam > 7.5$$

where all LTM (sphere diameter) values and LBBM (box lengths) values are given in voxels. The voxel resolution was 2.5 microns. The value of 7.5 (FibDiam+e_{DIAM}) for the melt equation was determined by examining the histograms of the LTMDiam and MinBB. These histograms have a strong peak at the diameter of the fibers. The aperture datasets were made up of two different fiber sizes. The 7.5 value was chosen just greater than the peak of the largest fiber. The value of 2 voxels (eBB) in the touch equation requires that MidBB be ˜25% larger the average fiber diameter. Noise in the IsBondPoint dataset is cleaned up by removing connected clusters of IsBondPoint voxels that number less than 100. As discussed earlier, only interior points are assigned. The bond points are morphologically dilated by one voxel in all direction to account for surface voxels. Dilated voxels that fall outside the original fiber mask are removed. The volume percentage is the number of IsBondPoint labeled voxels divided by the total number of fiber voxels in the ROI.

### Identifying Film

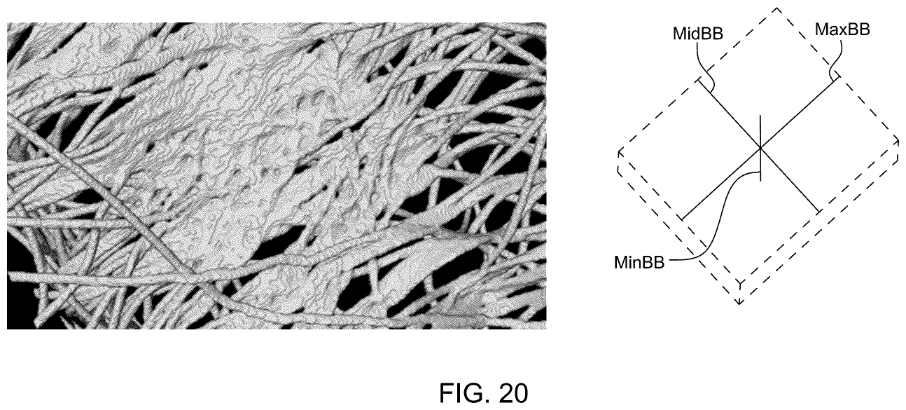

Film forms in a nonwoven when a multitude of fibers melt together to form a single thin plane of material. The LBBM can be used to detect other box like shapes in the nonwoven such as the film. FIG. 20 shows film that has formed at the bottom of a nonwoven embossment. The film has a thin flat shape similar to a thin flat box. A thin flat box will have a small minimum length (MinBB) and nearly equal maximum (MaxBB) and middle lengths (MidBB). Film voxels can be identified by the equation MaxBB<MidBB+eFilm.

Each interior point of the fibers is assigned a true or false value based on the film equation (MaxBB<MidBB+eFilm) as discussed above.

$$IsFilmPoint = MaxBB < MidBB + 4$$

where the LBBM (box lengths) values are given in voxels. The MaxBB values can reach a maximum length of 25 voxels. Requiring the MidBB to be within 4 voxels of MaxBB allows for a variation of 20% or less of the two lengths. Noise in the IsFilmPoint dataset is cleaned up by removing connected clusters of IsFilmPoint that number less than 100. The film points are morphologically dilated by one voxel in all directions to account for surface voxels. Dilated voxels that fall outside the original fiber mask are removed. The volume percentage is the number of film labeled voxels divided by the total number of fiber voxels.

The bond minus film volume percentage is calculated from a third dataset that was created from the final IsBondData and IsFilmData. Specifically, the final IsFilmData voxels are remove from the final IsBondData voxels and the percentage is calculated.

### Examples/Combinations

1. An air through bonded nonwoven material comprising:
a plurality of fibers;
a first plurality of areas formed in the fibers and having a first fiber to fiber bond density; and
a second plurality of areas formed in the fibers and having a second fiber to fiber bond density;
wherein the first plurality of areas do not overlap with the second plurality of areas;
wherein the first fiber to fiber bond density is greater than the second fiber to fiber bond density;
wherein the first plurality of areas are substantially free of a film; and
wherein the second plurality of areas are substantially free of a film.

2. The air through bonded nonwoven of Paragraph 1, wherein the first plurality of areas are locally densified areas formed in the plurality of fibers, and wherein at least some of the first plurality of areas are surrounded by at least some of the second plurality of areas.

3. The air through bonded nonwoven of Paragraph 1 or 2, wherein a ratio of the first fiber to fiber bond density to the second fiber to fiber bond density is in the range of about 1.2 to 10.

4. The air through bonded nonwoven material of any one of the preceding paragraphs, wherein the plurality of fibers comprise continuous spun fibers.

5. The air through bonded nonwoven material of any one of the preceding paragraphs, wherein the plurality of fibers comprise carded fibers.

6. The air through bonded nonwoven material of any one of the preceding paragraphs, wherein the first plurality of areas have a first basis weight, wherein the second plurality of areas have a second basis weight, and wherein the first basis weight is different than the second basis weight by at least 5 gsm.

7. The air through bonded nonwoven material of any one of the preceding paragraphs, wherein the first plurality of areas have a first basis weight, wherein the second plurality of areas have a second basis weight, and wherein the first basis weight is less than the second basis weight, and wherein the first and second basis weight are both greater than zero.

8. An air through bonded nonwoven material comprising:
a plurality of fibers;
a plurality of apertures defined in the plurality of fibers;
aperture rings surrounding the apertures; and
a land areas surrounding the aperture rings;
wherein the aperture rings have a first fiber to fiber bond density;
wherein the land areas have a second fiber to fiber bond density; and
wherein the first fiber to fiber bond density is greater than the second fiber to fiber bond density.

9. The air through bonded nonwoven material of Paragraph 8, wherein the plurality of fibers comprise continuous spun fibers.

10. The air through bonded nonwoven material of Paragraph 8, wherein the plurality of fibers comprise carded fibers.

11. The air through bonded nonwoven material of any one of Paragraphs 8-10, wherein the aperture rings have a fiber to fiber bond density about 1.2 times to about 10 times greater than a fiber to fiber bond density of the land areas.

12. The air through bonded nonwoven material of any one of Paragraphs 8-11, wherein the aperture rings have a higher basis weight than the land areas.

13. The air through bonded nonwoven material of any one of Paragraphs 8-12, wherein the nonwoven material comprises a non-apertured first region having a first basis weight and a non-apertured second region having a second basis weight, and wherein the first basis weight is different than the second basis weight by at least 5 gsm.

14. The air through bonded nonwoven material of any one of Paragraphs 8-13, wherein the aperture rings comprising the periphery of the apertures and tails of the apertures.

15. A method of producing a nonwoven material comprising:
conveying unconsolidated fibers;
creating locally densified areas or apertures comprising aperture rings in the unconsolidated fibers;
air through bonding the nonwoven material to create a greater fiber to fiber bond density in the locally densified areas or aperture rings compared to a fiber to fiber bond density in areas without the locally densified areas or the apertures comprising aperture rings; and
winding or slitting the nonwoven material.

16. The method of Paragraph 15, wherein the fiber to fiber bond density in the locally densified areas or aperture rings is about 1.2 times to about 10 times more than the fiber to fiber bond density in the areas without the locally densified areas or the aperture rings.

17. The method of Paragraph 15 or 16, wherein the unconsolidated fibers comprise continuous spun fibers.

18. The method of any one of Paragraphs 15-17, wherein the unconsolidated fibers comprise carded fibers.

19. The method of any one of Paragraphs 15-18, wherein the aperture rings have a higher basis weight compared to areas without the aperture rings.

20. The method of any one of Paragraphs 15-19, comprising using fluid jets to create the apertures comprising aperture rings. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited herein, including any cross referenced or related patent, patent publication, or patent application, is hereby incorporated by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, those of skill in the art will recognize that various other changes and modifications can be made without departing from the spirit and scope of the invention. It is therefore intended to cover in the appended claims all such changes and modifications that are within the scope of the present disclosure.

What is claimed is:

1. An air through bonded nonwoven material comprising:
a plurality of fibers;
a first plurality of areas formed in the fibers and having a first fiber to fiber bond density; and
a second plurality of areas formed in the fibers and having a second fiber to fiber bond density;
wherein the first plurality of areas do not overlap with the second plurality of areas;
wherein the first fiber to fiber bond density is greater than the second fiber to fiber bond density;
wherein the first plurality of areas are substantially free of a film; and
wherein the second plurality of areas are substantially free of a film.

2. The air through bonded nonwoven of claim 1, wherein the first plurality of areas are locally densified areas formed in the plurality of fibers, and wherein at least some of the first plurality of areas are surrounded by at least some of the second plurality of areas.

3. The air through bonded nonwoven of claim 2, wherein a ratio of the first fiber to fiber bond density to the second fiber to fiber bond density is in the range of about 1.2 to 10.

4. The air through bonded nonwoven material of claim 1, wherein the plurality of fibers comprise continuous spun fibers.

5. The air through bonded nonwoven material of claim 1, wherein the plurality of fibers comprise carded fibers.

6. The air through bonded nonwoven material of claim 1, wherein the first plurality of areas have a first basis weight, wherein the second plurality of areas have a second basis weight, and wherein the first basis weight is different than the second basis weight by at least 5 gsm.

7. The air through bonded nonwoven material of claim 1, wherein the first plurality of areas have a first basis weight, wherein the second plurality of areas have a second basis weight, and wherein the first basis weight is less than the second basis weight, and wherein the first and second basis weight are both greater than zero.

8. An air through bonded nonwoven material comprising:
a plurality of fibers;
a plurality of apertures defined in the plurality of fibers;
aperture rings surrounding the apertures; and
a land areas surrounding the aperture rings;
wherein the aperture rings have a first fiber to fiber bond density;
wherein the land areas have a second fiber to fiber bond density; and
wherein the first fiber to fiber bond density is greater than the second fiber to fiber bond density.

9. The air through bonded nonwoven material of claim 8, wherein the plurality of fibers comprise continuous spun fibers.

10. The air through bonded nonwoven material of claim 8, wherein the plurality of fibers comprise carded fibers.

11. The air through bonded nonwoven material of claim 8, wherein the aperture rings have a fiber to fiber bond density about 1.2 times to about 10 times greater than a fiber to fiber bond density of the land areas.

12. The air through bonded nonwoven material of claim 8, wherein the aperture rings have a higher basis weight than the land areas.

13. The air through bonded nonwoven material of claim 8, wherein the nonwoven material comprises a non-apertured first region having a first basis weight and a non-apertured second region having a second basis weight, and wherein the first basis weight is different than the second basis weight by at least 5 gsm.

14. The air through bonded nonwoven material of claim 8, wherein the aperture rings comprising the periphery of the apertures and tails of the apertures.

15. A method of producing a nonwoven material comprising:
conveying unconsolidated fibers;
creating locally densified areas or apertures comprising aperture rings in the unconsolidated fibers;
air through bonding the nonwoven material to create a greater fiber to fiber bond density in the locally densified areas or aperture rings compared to a fiber to fiber bond density in areas without the locally densified areas or the apertures comprising aperture rings; and
winding or slitting the nonwoven material.

16. The method of claim 15, wherein the fiber to fiber bond density in the locally densified areas or aperture rings is about 1.2 times to about 10 times more than the fiber to fiber bond density in the areas without the locally densified areas or the aperture rings.

17. The method of claim 16, wherein the unconsolidated fibers comprise continuous spun fibers.

18. The method of claim 15, wherein the unconsolidated fibers comprise carded fibers.

19. The method of claim 15, wherein the aperture rings have a higher basis weight compared to areas without the aperture rings.

20. The method of claim 15, comprising using fluid jets to create the apertures comprising aperture rings.
